(19) United States
(12) Patent Application Publication
(10) Pub. No.: US 2011/0223222 A1
(43) Pub. Date: Sep. 15, 2011

(54) Antiperspirant Compositions and Methods for Reducing Perspiration in Humans

(76) Inventors: Alexandros Spyros Botsaris, Rio de Janeiro RJ (BR); Cristina Dislich Röpke, Tremembe (BR); Timothy J. Balts, Morumbi (BR); Marcelo Simioni Pontes, Bonfiglioli (BR); Paulo Roberto Francischini de Carvalho, Sao Paulo-SP (BR); Tânia Sawada, Vila Indiana (BR)

(21) Appl. No.: 13/058,564

(22) PCT Filed: Aug. 11, 2009
(86) PCT No.: PCT/BR09/00244

§ 371 Date: May 26, 2011

(30) Foreign Application Priority Data
Aug. 11, 2008 (FR) 08/04556

(51) Int. Cl. A61K 8/11; A61K 8/34; A61K 8/49; A61K 8/35; A61K 8/97; A61Q 15/00

(52) U.S. Cl. 424/401; 424/65

(57) ABSTRACT

A new method for inhibiting or reducing perspiration and preventing body unpleasant odors is provided. Alternative antiperspirant actives capable of activating TRPM8 channels are topically applied, leading to inhibition or reduction of sweat production. Preferred agent is a TRPM8 agonist, derived or not from essential oils of plants. Cosmetic or pharmaceutical product comprising the TRPM8 agonist and the use of TRPM8 agonists as antiperspirant active are also claimed.

### FIELD OF THE INVENTION

The present invention is applied to the fields of Cosmetics, Pharmacy and Medicine. It relates to the use of TRPM8 agonists or plant essential oils containing TRPM8 agonists, capable of reducing or inhibiting perspiration and/or unpleasant body odors when topically applied. Antiperspirant products comprising the TRPM8 agonists or plant essential oils that present TRPM8 agonists as a component, as well as a new method for the inhibition or reduction of the human perspiration and/or body unpleasant odors are also described.

### BACKGROUND OF THE INVENTION

Many topical antiperspirant products are commercially available in a variety of formulations and product forms. These products typically contain a solid and/or liquid carrier in combination with an antiperspirant active.

Nowadays, commercially available antiperspirant actives are limited mostly to astringent metal (zirconium and aluminum) salts, which are intended to prevent, or at least control, perspiration on the surface of the skin, particularly on the axillae.

The most accepted theory of effectiveness of these actives is that such metal salts antiperspirants work by physically blocking the sweat duct through the formation of a plug composed by insoluble metal salts, which precipitate within the duct. After topical application, the salts dissolve in sweat, diffuse into the sweat ducts where the pH of the sweat causes the salt to precipitate, thereby filling the duct and resulting in the blockage of the perspiration flow. It has also been suggested that said plugs form an obstructive conglomerate that may cause damage to luminal epithelial cells. Therefore, although the secretory portion of the sweat gland is not affected by the metallic salts, the long-term blockage of the duct may lead to functional and structural degeneration of the sweat gland with loss of secretory function (Benohanian, (2001) Clinics in Dermatology 19: 398-405).

Thus, it is not surprising that some disadvantages are often associated with the use of such metal salt antiperspirants. For example, it can be skin unfriendly, leading to some degree of skin irritation. They may also leave a white residue on the skin that is generally unsightly and sticky. Also, such antiperspirants often leave yellow stains on fabric as a result of the reaction between the sweat and the salt, which may be difficult to remove.

Further, the metal salt antiperspirants may not be fully effective. At best, the perspiration inhibition of available commercial products ranges from 20-50%, depending on form and active ingredient. Efficacy may be further limited, especially if the user decides to reduce the amount used or frequency of application to lessen irritation and/or fabric damage.

These adverse effects may have an influence on whether or not to use antiperspirants thereby depriving some potential consumers of such cosmetics. Hence, there is a clear need for a truly effective antiperspirant that is aesthetically pleasing, non-irritating to skin and non-damaging to clothing.

In this view, other alternative actives have been investigated for antiperspirant efficacy. The use of numerous “non-conventional” antiperspirant or deodorant agents is described in the prior art. For example, the use of anticholinergic materials, including scopolamine derivatives, is described in U.S. Pat. No. 3,312,709 (MacMillan); U.S. Pat. No. 4,022,787 (Soldati et al.); and a series of patents by Felger et al. (U.S. Pat. No. 4,517,176; U.S. Pat. No. 4,546,096; U.S. Pat. No. 4,720,494). However, because of their poor skin-penetration properties, they require injections for effectiveness. Also, substances capable of inhibiting calcium influx have been proposed to be used as antiperspirant actives since they were capable of reducing sweat production from sweat glands (WO2002/11690, Burry and Evans). Aldehydes, such as formaldehyde, can suppress perspiration, but their sensitization potential has halted any attempt to put them on the market. Antiadrenergics and metabolic inhibitors have been studied academically. All these materials are claimed to reduce perspiration at its origin, that is, they control perspiration acting on the sweat glands.

Still, other alternative antiperspirant actives include waterinsoluble, occlusive, film-forming polymers when applied topically to the skin. Examples of such antiperspirant polymers are described, for example, in U.S. Pat. No. 5,508,024 (Tranner); U.S. Pat. No. 6,106,813 (Mondet et al.) and U.S. Pat. No. 5,626,856 (Berndt). However, it has been found that the application of these films does not always improve antiperspirant efficacy as compared to the application of commercially available antiperspirant products containing conventional zirconium and aluminum salts.

The present invention relates to the reduction or inhibition of perspiration through a physiological mechanism mediated by the transient receptor potential (TRP) channels. The TRP receptor family comprises more than 30 cation channels and can be divided into seven main subfamilies. The TRP channels and related TRP-like receptors connote sensory responsivity to the entire continuum of thermal exposure, selectively responding to threshold temperatures ranging from noxious hot through noxious cold as well as to certain chemicals that mimic these sensations. There are two genes, TRPM8 (McKemy et al. (2002) Nature 416: 52-58) and TRPA1 (Story et al. (2003) Cell 112: 819-829; McKemy (2005) Molecular Pain 1: 16) which are known to be cold-sensing TRP channels. Specifically, TRPM8, also called CMR1, is known to be stimulated by cold temperatures as well as by cooling compounds, such as menthol, icilin, menthone, eucalyptol, geraniol and linalool (McKemy et al. (2002) Nature 416: 52-58; Peier et al. (2002) Cell 108: 705-715; Brauchi et al. (2004) PNAS 10: 15494-15499; Voets et al. (2004) Nature 430: 748-754).

The TRPM8 channel is expressed in a subset of temperature-sensing small dorsal root and trigeminal ganglion neurons, and thus has been associated with sensing cold temperatures at mammalian thermoreceptor nerve endings. In addition to its presence on sensory neurons, functional TRPM8 mRNA expression was reported in a variety of organs or tissues, such as the brain, lung, bladder, gastrointestinal tract, blood vessels and prostate, providing the possibility for therapeutic modulation in a wide range of disease states or conditions. (See, for example, International Patent Applications WO 2004/054497 (Plath et al.); WO 2007/053570 (Qin and Flores); WO 2008/015403 (Fleetwood-Walker et al.); WO 2006/029142 (Gu)).

At present, there is not an antiperspirant active, composition or formulation which could successfully substitute aluminum and zirconium salts, capable of reducing or inhibiting perspiration through the activation of a cold-sensing receptor, which is the essence of the present invention. The use of menthol or eucalyptol, always combined with metal salt antiperspirants in antiperspirant and/or deodorant formulations is described, but it aims at simply providing the user with a cooling and refreshing sensation or a deodorant effect (see, for example, U.S. Pat. No. 6,793,915 (Guenin et al.), JP2002037722A2 (Fujihira)).

It is already known form prior art, for example, a refreshing composition comprising menthol or mint in concentrations of 0.05 or 0.5%. Those compositions, however, are useful only as deodorants due to the odor masking characteristic and refreshing sensation provided by the actives. Those molecules have low molecular weight and evaporates rapidly, thus leading to said momentary refreshing feeling, but do not present an actual antiperspirant effect.

Examples of compositions wherein menthol or menthe oil are used just as deodorizing and/or refreshing active ingredients are disclosed in JP 2003073248, US 2008124282, US 2004076694 and US 2004076696.

Thus, it is believed that the use of a TRPM8 agonist as an antiperspirant active or the antiperspirant effect caused by its action on a TRPM8 receptor is neither suggested nor anticipated in these or in other prior art documents.

Thus, it is an object of the present invention to provide an alternative method of inhibiting or reducing perspiration, mediated by the mechanism of the TRPM8 receptors, with the use of TRPM8 agonists or plant essential oils containing TRPM8 agonists. It is further an object of the present invention to provide alternative antiperspirant actives and antiperspirant compositions with excellent efficacy, excellent cosmetic properties and aesthetics, reduced or no skin irritation and no fabric damage.

### SUMMARY OF THE INVENTION

In one aspect, the invention provides an antiperspirant composition for topical application to inhibit or reduce perspiration and/or unpleasant body odor comprising: a vehicle and a TRPM8 agonist, or a plant essential oil containing a TRPM8 agonist, or a combination thereof, wherein the TRPM8 agonist is present at weight per weight between 0.01% and about 30%, of the composition.

In one embodiment, the TRPM8 agonist is selected from the group consisting of menthol, eucalyptol, eugenol, geraniol, linalool, nerolidol, terpineol, carvone, menthone, pulegone or carvacrol, or a combination thereof.

In another embodiment, the TRPM8 agonist is menthol or eucalyptol or a combination thereof.

In other embodiment, the plant essential oil containing a TRPM8 agonist is Mentha sp. or Eucalyptus sp. essential oils.

In a further embodiment, the TRPM8 agonist is at weight per weight between 0.02% and about 20%, or 0.05% and about 15%, 0.1%, or about 10%, 0.2% or about 5% of the composition.

In another further embodiment, the vehicle comprises a material selected from the group consisting of water, alcohol, an anhydrous solution and a hydrophobic matrix.

In another aspect, the invention provides a method for inhibiting or reducing perspiration and/or body unpleasant odors in a human comprising a step of topically applying an antiperspirant composition comprising a TRPM8 agonist, a plant essential oil containing a TRPM8 agonist, or a combination thereof.

In a further aspect, the invention provides the use of a TRPM8 agonist, or a plant essential oil containing a TRPM8 agonist, or a combination thereof, for the manufacture of an antiperspirant product for inhibiting or reducing perspiration and/or unpleasant body odors.

### BRIEF DESCRIPTION OF THE DRAWINGS

FIG. 1 presents the antiperspirancy efficacy of the antiperspirant compositions comprising Eucalyptus globulus essential oil, Mentha arvensis essential oil or menthol, in one hour panel, which showed a statistically significant reduction in sweat production of 63.5%±22.5, 70.7%±22.1 and 61.4%±23.1, respectively.

FIG. 2 presents the antiperspirancy efficacy of the antiperspirant compositions comprising Eucalyptus globulus essential oil, Mentha arvensis essential oil or menthol, in two hours panel, which showed a statistically significant reduction in sweat production of 68.5%±1.8, 68.4%±17.4 and 63.7%±21.8, respectively.

FIG. 3 presents the antiperspirancy efficacy of the antiperspirant compositions comprising Eucalyptus globulus essential oil, Mentha arvensis essential oil or menthol, in four hours panel, which showed a statistically significant reduction in sweat production of 68.5%±18.8, 68.4%±17.4 and 63.7%±21.8, respectively.

FIG. 4 shows the percentage of reactive volunteers for antiperspirant compositions comprising Eucalyptus globulus essential oil, Mentha arvensis essential oil or menthol.

### DETAILED DESCRIPTION OF THE INVENTION

Methods and compositions or formulations comprising a TRPM8 agonist or plant essential oils containing TRPM8 agonists are provided herein for inhibiting or reducing perspiration in humans and/or preventing unpleasant body odors by topical administration. Such perspiration typically occurs in areas such as the axillae, feet, palms, back, face, neck, groin and other more sweaty parts of the body. For the purposes of the present invention, the terms “composition” and “formulation” are used interchangeably.

A preferred embodiment comprises topically applying to such sweaty parts of the body, more preferably in the axillae, an antiperspirant composition comprising a TRPM8 agonist or plant essential oils containing TRPM8 agonists that activate the TRPM8 channels, producing an antiperspirant effect. Superior efficacy is accomplished as the desired antiperspirancy does not rely on physical plugs filling in pores of the skin, although a synergistic effect may be accomplished by plugging the pores, as discussed below.

The product formulation may vary depending on what is appropriate or desired for the form. Such cosmetic antiperspirant products may be available in a variety of product forms, for example as sticks, powders, roll-on suspensions or lotions, emulsions, gels, creams, aerosols, pump and squeeze sprays formulations. It also may be anhydrous or water based, utilizing such combination of components as to provide the desired profile of dose and aesthetics. In general, it will include a vehicle/carrier, dispersant, emollient, fragrance, surfactant and structurants. Besides the use in the manufacture of antiperspirants products for inhibition or reduction of perspiration in the axillae, the TRPM8 agonist may also be incorporated into a reservoir or other type of “patch” or plaster or embedded in a matrix for controlled release in other sweaty parts of the body, for example, but not limited to, sole insert to deliver to foot, or sock or glove to deliver to sole/palm. Also, the TRPM8 agonist may be incorporated into cosmetic or pharmaceutical products, e.g., moisturizing creams and body sprays.

The antiperspirant composition may comprise a vehicle, and at least one TRPM8 agonist at percentages ranging from 0.01% to 30% by weight relative to the total weight of the composition (w/w), with a preferred range of 0.02% to 20%, more preferred ranges of 0.05% to 15%, 0.1 to 10% and the most preferred range of 0.2% to 5%. It is evident that the skilled in the art is enabled to determine the more appropriate w/w percentage of TRPM8 agonist or essential oil containing TRPM8 agonist to be included in an antiperspirant composition, according to scientific studies and regulation guidelines, to guarantee the best antiperspirancy effect together with the product safety.

The term “TRPM8 agonists” as used herein relates to natural or synthetic compounds that bind or preferentially bind to and activate TRPM8 channels, including for example, eucalyptol, menthol, eugenol, icilin and analogs thereof, capable of inhibiting or reducing perspiration when topically applied. Such perspiration inhibition or reduction is site-directed, that is, it is restricted to the local where the TRPM8 agonist or a composition comprising a TRPM8 agonist, is applied. Other examples of TRPM8 agonists include, but are not limited to, terpenes, e.g. cyclic terpenes, such as eucalyptol, menthol and menthyl lactate (Frescolat ML), and derivatives or enantiomers thereof.

Terpenes are a class of organic compounds found, for example, in essential oils and have been employed as fragrances, flavorings and medicines. A terpene refers to a compound that is based on an isoprene unit (C₅H₈) and can be classified based on the number of isoprenoid unit that they contain. For example, a monoterpene consists of two isoprene units (C10), sesquiterpenes have three (C15) and diterpenes have four (C20). A commonly used terpene is menthol, which has been incorporated into inhalation compositions, emollient cosmetics and food preparations. Other examples of terpenes that can act as TRPM8 agonist include, but are not limited to, eucalyptol (1,8-cineole), geraniol, nerolidol, menthone, cineole, terpineol, D-limonene, linalool, pulegol (e.g., isopulegol), pulegone, carvone and carvacrol. Other examples of terpene and non-terpene TRPM8 agonists include Trans-p-menthane-3,8-diol, cis-p-menthane-3,8-diol, L-carvone (Spearmint oil), N,2,3-trimethyl-2-isopropylbutanamine (WS-23), N-ethyl paramenthane-3-carboxaminde (WS-3), menthone glycerin acetal (Frescolat MGA), menthoxypropanediol (Cooling agent 10), Coolact P, PMD-38, monomenthyl succinate (Physcool), monomenthyl glutarate and hydroxycitronellal, (see for example, Behrendt et al., (2004) British Journal of Pharmacology 141: 737-745; Calixto et al., (2005) Pharmacology & Therapeutics 106: 179-208; Erman et al., (2005) Cosmetics & Toiletries 120: 105-118, the contents of which are incorporated hereinto by reference).

For the purposes of this invention, an essential oil from a plant or botanical source may also be used to manufacture a cosmetic or pharmaceutical product, such as an antiperspirant product, as long as it comprises a component that binds or preferentially binds to and activates a TRPM8 channel, i.e., a TRPM8 agonist, leading to inhibition or reduction of perspiration when topically applied. For the purposes of this invention, an essential oil is a predominantly volatile material or materials isolated by some physical or chemical process from an odorous botanical source. The oils extracted by the physical process can contain some non-volatile material, as is well known in the art. Since the individual components of the essential oils are the individual compounds that will interact with the TRPM8 channels in the manner described herein, it is apparent that essential oil components that are sufficiently active will also be useful in the practice of the invention.

In a preferred embodiment of the invention, the essential oil is derived from Eucalyptus sp. (e.g. Eucalyptus globulus) and Mentha sp. (e.g., Mentha arvensis), which presents eucalyptol (1,8-cineole) and menthol, respectively. Additional botanical genera and plant species whose essential oils contain a component that act as a TRPM8 agonist include, but are not limited to, Achillea sp. (e.g., A. fraasii, A. holosericea, A. setacea, A. taygetea, A. teretifolia), Aframomum sp. (e.g., A. stipulatum), Artemisia sp. (e.g., A. haussknechtii), Calamintha sp. (e.g., C. sylvatica), Cymbopogon sp. (e.g. C. citratus), Eucalyptus sp. (e.g. E. alba, E. camadulensis, E. citriodora, E. deglupta, E. globulus, E. robusta, E. saligna, E. terticornis), Lippia sp. (e.g., L. graveolens), Melalleuca sp. (e.g., M. leucadendron), Nepeta sp. (e.g., N. argolica, N. camphorata, N. crispa), Ocimum sp. (e.g., O. americanum, O. gratissimum), Salvia sp. (e.g., S. officinalis, S. tomentosa), Tanacetum sp. (e.g., T. vulgare), Thymus sp. (e.g., T. lotocephalus, T. praecox, T. serpyllum, T. xmourae), Turnera sp. (e.g., T. diffusa), as non-limiting examples of plants presenting eucalyptol (1,8-cineole); Coriandrum sp. (e.g. C. sativum), Cymbopogon sp. (e.g., C. martini), Daucus sp. (e.g., D. carota), Lonicera sp. (e.g., L. japonica), Malus sp. (e.g., M. pumila), Melissa sp. (e.g., M. officinalis), Nepeta sp. (e.g., N. cataria), Ocimum sp. (e.g. O. basilicum, O. gratissimum), Pelargonium sp. (e.g., P. graveolens), Pinus sp. (e.g. P. radiata), Polianthes sp. (e.g., P. tuberosa), Rhodiola sp. (e.g., R. rosea), Rosa sp., Salvia sp. (e.g., S. lavandulaefolia), Satureja sp. (e.g., S. montana), Thea sp. (e.g., T. sinensis), Thymus sp. (e.g., T. vulgaris), Zanthoxylum sp. (e.g. Z. bungeanum, Z. schinifolium), as non-limiting examples of plants presenting geraniol; Achillea sp. (e.g., A. millefolium), Actinidia sp. (e.g., A. macrosperma), Aloysia sp. (e.g., A. triphylla), Aniba sp. (e.g., A. rosaeodora), Anthemis sp. (e.g., A. nobilis), Artemisia sp. (e.g., A. abrotanum, A. absinthium, A. annua, A. scoparia, A. sieberi), Bursera sp. (e.g., B. aloexylon, B. lancifolia), Cinnamomum sp. (e.g., C. iners, C. osmophloeum), Croton sp. (e.g., C. cajucara), Cymbopogon sp. (e.g., C. martini, C. winterianus), Cyperus sp. (e.g., C. articulatus, C. rotundus), Filipendula sp. (e.g., F. vulgaris), Grammosciadium sp. (e.g., G. platycarpum), Hedychium sp. (e.g., H. arsenic). Laurus sp. (e.g., L. nobilis), Lavandula sp. (e.g., L. angustifolia, L. hybrida, L. officinalis), Lippia sp. (e.g., L. alba, L javanica), Melissa sp. (e.g., M. officinalis), Monarda sp. (e.g., M. didyma), Myrica sp. (e.g., M. gale), Myrtus sp. (e.g., M. communis), Ocimum sp. (e.g., O. basilicum, O. sanctum), Origanum sp. (e.g., O. vulgare), Plantago sp. (e.g., P. asiatica), Pseudopanax sp., Sassafras sp. (e.g., S. albidum), Satureja sp. (e.g., S. parnassica), Strychnos sp. (e.g., S. spinosa), Thymbra sp. (e.g., T. capitata), Thymus sp. (e.g., T. albicans, T. carnosus, T. mastichina, T. vulgaris), Zanthoxylum sp. (e.g., Z. armatum, Z. bungeanum, Z. rhoifolium, Z. schinifolium), Zataria sp. (e.g., Z. multiflora), as non-limiting examples of plants presenting linalool; Angelica sp. (e.g., A. glauca), Baccharis sp. (e.g., B. dracunculifolia), Comptonia sp. (e.g., C. peregrina), Discaria sp. (e.g., D. americana), Echinodorus sp. (e.g., E. grandiflorus), Eryngium sp. (e.g., E. comiculatum), Melaleuca sp., Piper sp. (e.g., P. gaudichaudianum), Pittosporum sp. (e.g., P. tobira), Pseudopanax sp., Stachys sp., Strychnos sp. (e.g., S. spinosa), Thymus sp. (e.g., T. serpyllum), Virola sp. (e.g., V. surinamensis), Zanthoxylum sp. (e.g., Z. hyemale, Z. rhoifolium, Z. setulosum), as non-limiting examples of plants presenting nerolidol; Abies sp. (e.g., A. coreana), Achillea sp. (e.g., A. millefolium), Aframomum sp. (e.g., A. danieffi, A. stipulatum), Alpinia sp. (e.g., A. galanga), Artemisia sp. (e.g., A. absinthium, A. feddei, A. haussknechtii, A. lavandulaefolia), Bursera sp., Cinnamomum sp. (e.g., C. osmophleum, C. zeylanicum), Citrus sp., Cryptomeria sp. (e.g., C. japonica), Cymbopogon sp. (e.g., C. citratus), Discaria sp. (e.g., D. americana), Dracocephalum sp. (e.g., D. kotschyi), Drimys sp. (e.g., D. granatensis), Eucalyptus sp. (e.g., E. alba, E. camadulensis, E. citriodora, E. deglupta, E. globulus, E. propinqua, E. robusta, E. saligna, E. terticornis, E. urophylla), Eugenia sp. (e.g., E. dysenterica), Heteropyxis sp. (e.g., H. dehniae), Houttuynia sp. (e.g., H. cordata), Juniperus sp. (e.g., J. procera), Laurus sp. (e.g., L. nobilis), Lavandula sp. (e.g., L. angustifolia, L. viridis), Lippia sp. (e.g., L. multiflora), Majorana sp. (e.g., M. hortensis), Melaleuca sp. (e.g., M. alternifolia), Myrica sp. (e.g., M. gale), Ocimum sp. (e.g., O. americanum, O. gratissimum), Origanum sp. (e.g., O. cordifolium), Pelargonium sp., Peucedanum sp. (e.g., P. ostruthium), Pimenta sp. (e.g., P. racemosa), Pistacia sp., Rhododendron sp. (e.g., R. tomentosum), Salvia sp. (e.g., S. lavandulaefolia, S. sclarea), Shorea sp. (e.g., S. robusta), Tanacetum sp. (e.g., T. vulgare), Tetradenia sp. (e.g., T. riparia), Thymus sp. (e.g., T. caespititius, T. kotschyanus, T. persicus, T. vulgaris), Zanthoxylum sp. (e.g., Z. bungeanum, Z. schinifolium), Zea sp. (e.g., Z. mays), Zingiber sp. (e.g., Z. roseum), as non-limiting examples of plants presenting terpineol; Aloysia sp. (e.g., A. polystachya), Anethum sp. (e.g., A. graveolens), Artemisia sp. (e.g., A. alba), Balsamita sp. (e.g., B. major, B. suaveolens), Carum sp. (e.g., C. carvi), Dracocephalum sp. (e.g., D. grandiflorum, D. renati, D. ruyschiana), Lippia sp. (e.g., L. alba, L. javanica), Mentha sp. (e.g, M. piperita, M. spicata, M. suaveolens), Nigella sp. (e.g., N. sativa), Ocimum sp. (e.g., O. basilicum), Origanum sp. (e.g., O. majorana), as non-limiting examples of plants presenting carvone; Achyrocline sp. (e.g., A. satureioides), Lavandula sp. (e.g., L. stoechas), Magnolia sp. (e.g., M. biondii, M. denudata, M. sprengeri), Mentha sp. (e.g., M. aplocalyx, M. aquatica, M. citrata, M. crispa, M. gentilis, M. longifolia, M. longifolia, M. piperita, M. rotundifolia, M. suaveolens), Minthostachys sp. (e.g., M. verticillata), Perilla sp. (e.g., P. frutescens), Schizonepeta sp. (e.g., S. tenuifolia), as non-limiting examples of plants presenting menthol; Bystropogon sp. (e.g., B. canariensis, B. origanifolius, B. plumosus, B. wildpretii), Cunila sp. (e.g., C. polyantha), Lavandula sp. (e.g., L. stoechas), Melissa sp. (e.g., M. officinalis), Mentha sp. (M. aplocalyx, M. aquatica, M. arvensis, M. crispa, M. gentilis, M. longifolia, M. piperita, M. pulegium, M. rotundifolia, M. sachalinensis, M. suaveolens), Minthostachys sp. (e.g., M. mollis, M. verticillata), Pelargonium sp. (e.g., P. tomentosum), as non-limiting examples of plants presenting menthone; Cunila sp. (e.g., C. polyantha), Lavandula sp. (e.g., L. stoechas), Mentha sp. (e.g, M. arvensis, M. cervina, M. crispa, M. microphylla, M. piperita, M. pulegium, M. spicata, M. suaveolens), Micromeria sp. (e.g., M. cilicica), Minthostachys sp. (e.g., M. mollis, M. verticillata), Origanum sp. (e.g., O. majorana), Poliomintha sp. (e.g. P. incana), Salvia sp. (e.g., S. rhytidea), Schizonepeta sp. (e.g., S. tenuifolia), Thevetia sp. (e.g., T. peruviana), Ziziphora sp. (e.g., Z. clinopodioides, Z. persica, Z. taurica), as non-limiting examples of plants presenting pulegone; Arnica sp. (e.g., A. longifolia), Aster sp. (e.g, A. hesperius), Carum sp. (e.g., C. copticum), Coridothymus sp. (e.g., C. capitatus). Lantana sp. (e.g., L. achyranthifolia), Lavandula sp. (e.g., L. multifida), Lippia sp. (e.g., L. graveolens, L. multiflora, L. sidoides), Majorana sp. (e.g., M. syriaca), Micromeria sp. (e.g., M. nervosa), Monarda sp. (e.g., M. didyma), Mosla sp. (e.g., M. chinensis), Nigella sp. (e.g., N. sativa), Origanum sp. (e.g., O. acutidens, O. compactum, O. cordifolium, O. minutiflorum, O. onites, O. sativum, O. virens, O. vulgare), Plectranthus sp. (e.g., P. cylindraceus), Satureja sp. (e.g., S. cuneifolia, S. hortensis, S. khuzistanica, S. laxiflora, S. montana, S. parnassica, S. thymbra, S. wiedemanniana), Stachys sp. (e.g., S. persica), Thymbra sp. (e.g., T. capitata, T. spicata), Thymus sp. (e.g., T. caespititius, T. eigii, T. kotschyanus, T. longicaulis, T. pectinatus, T. persicus, T. pulegioides, T. revolutus, T. serpyllum, T. vulgaris, T. zygioides), Zataria sp. (e.g., Z. multiflora), as non-limiting examples of plants presenting carvacrol. (See, for example, Behrendt et al., (2004) British Journal of Pharmacology 141: 737-745; Sonboli et al., (2006) Z. Naturforsch 61:160-164; Unlü et al., (2002) J. Ethnopharmacol. 83:117-121; Salgueiro et al., (2000) Biochem. Syst. Ecol. 28:457-470; Jalali Heravi et al. (2007) J. Chromatogr. A. 1160:81-89; Chiang et al., (2005) Clin. Exp. Pharmacol. Physiol. 32:811-816; Thompson et al., (2003) J. Chem. E-col. 29:859-880; Beytía et al., (1969) Arch. Biochem. Biophys. 129:346-356; Radulović et al., (2007) Fitoterapia 78:565-570; Jia et al. (1999) Arch. Biochem. Biophys. 372:143-149; Shafiee and Javidnia (1997) Planta Med. 63:371-372; Klopell et al. (2007), Z Naturforsch 62:537-542; Hoet et al. (2006) Planta Med. 72:480-482; Simionatto et al. (2005) Planta Med. 71:759-763, Cha et al, (2007) J Microbiol Biotechnol. 17:2061-2065; Jalali and Sereshti (2007) J Chromatogr A. 1160:81-89; Prakash et al. (2006) J Ethnopharmacol. 106:344-347; Lota et al. (2002) J Agric Food Chem. 50:796-805; Goren et al. (2002) Z Naturforsch 57:797-800, contents of which are incorporated hereinto by reference).

It is clear that a person skilled in the art is enabled to identify and select other plant genera or species that could be used as sources of essential oils or individual components thereof suitable for the present invention, without departing from the spirit and scope of the present invention. Also, the skilled in the art is enabled to determine the components (including TRPM8 agonist) and concentrations thereof in a chosen essential oil, for example, via techniques including, but not limited to, HPLC, GC, GC-MS, GC-MS-MS, LC-MS, LC-NMR, LC-DAD, LC-MS-MS. Thus, the skilled in the art may determine the amount of essential oil to be used in the manufacture of the antiperspirant composition, in order to result in appropriate percentages of TRPM8 agonist(s) in said composition.

The formulation may further include more than one TRPM8 agonists, providing a synergistic effect. The term “synergistic effect” is defined as the effect achieved when two or more antiperspirant actives, for example, two or more TRPM8 agonists or a TRMP8 agonist and a metal salt antiperspirant, work together to produce a result not obtainable by any of the actives independently. In this sense, the combination of two or more TRMP8 agonists may result in an enhanced antiperspirant efficacy or lead to an increase in the duration of antiperspirancy. More preferably, the synergistic effect is achieved through the combination of menthol and eucalyptol. It is clear that a skilled person is enabled to select other combinations that may result in a synergistic effect in antiperspirancy, without departing from the spirit and scope of the invention.

The vehicle can be inert or can possess cosmetic, physiological and/or pharmaceutical benefits on its own. Vehicles may comprise water, alcohol, an anhydrous solution or a hydrophobic matrix and can be formulated with non-essential additional ingredients such as liquid or solid emollients, enhancers or modifiers, structurants, solvents, thickeners, surfactants, co-surfactants, fragrances, sensory modifiers, emulsifiers, gelling agents, dispersants, deodorant, suspending agents, wash-off agents, controlled release agents, skin penetration enhancers, controlling or release agents humectants, materials with keratolytic activity, antioxidant, anti-inflammatory, anti-fungal, anti-bacterial, UV-protection, skin conditioning, etc, or other activities which are intended to impart benefit to the skin itself or modify other functions of the sweat gland/sweat duct unit. Materials within a given functional group may be combined to balance the benefits. For example, low and high melt point waxes may be combined to optimize manufacturability. Likewise, polyethylene waxes may be used in combination with classic organic materials. Similarly, silicone and silicone derivatives may be combined to control and vary product properties.

It will be appreciated that the preferred embodiments of the antiperspirant compositions may also be incorporated with a metal salt antiperspirant to provide a synergistic combination. As examples and not by way of limitations, metal salt antiperspiration actives such as aluminum, zirconium, mixed aluminum/zirconium salts, and more, can be used. Antiperspirancy is thus accomplished by different mechanism of action, by both activating the-TRPM8 channels as well as blocking of the sweat ducts. In such cases, a lower concentration of salts may be employed.

It will be appreciated that the antiperspirant methods and compositions of the invention provide several advantages over conventional antiperspirants. A pleasant aesthetic touch is provided, as the preferred embodiments do not leave a sticky, tacky, oily or greasy feeling. It will further be appreciated that the preferred embodiments do not irritate the skin. No residues result from using the preferred embodiments. Instead, the preferred embodiments go on to the target body area clearly without whitening the skin or clothing.

Another embodiment of the present invention is the use of the microencapsulation and controlled release technology, well known in the art (See, e.g., U.S. Pat. No. 7,235,261 (Smith et al.); U.S. Pat. Appl. No. US20040091435 (Shefer and Shefer), the contents of which are incorporated hereinto by reference.). It is generally directed to encapsulating core materials in a protective covering until time of use. In a preferred embodiment, the core material is a substance that acts as a TRMP8 agonist. It is also preferred that the coating material be a water-sensitive matrix, such as starches, polysaccharides or mixtures thereof, so as to be releasable upon contact with water or aqueous solutions. Generally a coating material of this composition provides the most desirable time-release properties when used with the antiperspirant/deodorant of the invention, since the antiperspirant actives are released when perspiration occurs. Also, it is important that the coating material be of a composition that is compatible with the antiperspirant/deodorant formulation. Otherwise the microcapsule will be dissolved by the ingredients of the formulation prior to the purchase and use of the product by the consumer. Generally coating materials of a starch/polysaccharide constituency have excellent compatibility with antiperspirant/deodorant formulations.

Microcapsules having the desired complex wall structure to be used in the present invention can be conveniently made by coacervation processes as disclosed, for example, in U.S. Pat. No. 5,112,688, in which at least a major portion of the material to be encapsulated is converted to an emulsion having particle diameters of more than about 50 microns and another smaller portion of the same material, or a different material, or mixtures thereof, is converted to an emulsion or suspension having particle diameters of less than about 15 microns before encapsulation, e.g., the coacervation process uses an emulsion with a bimodal distribution. Such microcapsules made by coacervation processes can be prepared from gelatin and a polyanionic material, coated or not by a cationic polymer.

Fragrances, when present, typically comprise up to about 1% of the total product. Coloring agents and preservatives can be added as desired. The optional ingredients are other cosmetic adjuncts conventionally employed in antiperspirant or deodorant products. The components which can optionally be present in the composition carrier can conveniently form the balance of the composition. Propellants commonly employable in aerosol compositions herein comprise hydrocarbons (or much less desirably halohydrocarbons) having a boiling point of below 10° C. and especially those with a boiling point below 0° C. It is especially preferred to employ liquefied hydrocarbon gases, and especially C3 to C6 hydrocarbons, including propane, isopropane, butane, isobutane, pentane and isopentane and mixtures of two or more thereof. Preferred propellants are isobutane, isobutane/isopropane, isobutane/propane and mixtures of isopropane, isobutane and butane. Other propellants that can be contemplated include alkyl ethers, such as dimethyl ether or compressed non-reactive gasses such air, nitrogen or carbon dioxide.

The composition according to the invention can take any form of a product suited to or adapted for topical application to human skin, and is usually contained in a suitable holder or dispenser to enable it to be applied to the area of the skin, particularly the axilla, where control of perspiration and deodorancy is required.

Many alterations and modification may be made by those having ordinary skill in the art without departing from the spirit and scope of the invention. Therefore, it must be understood that the illustrated embodiments have been set forth only for the purposes of examples and that they should not be taken as limiting the invention as defined by the following claims. For example, notwithstanding the fact that the elements of a claim are set forth below in a certain combination, it must be expressly understood that the invention includes other combinations of fewer, more or different ones of the disclosed elements.

The present invention is further illustrated by the following specific examples. The examples are provided for illustration only and are not to be construed as limiting the scope or content of the invention in any way.

### EXAMPLES

### Example 1

### Antiperspirant Composition

| Ingredient | Final % |
|---|---|
| Water | 90-96 |
| TRPM8 agonist | 0.1-10 |
| Dimethicone | 0.1-0.9 |
| PEG-5 Cocoate, PEG-8 Dicocoate, Iodopropynyl | 0.05-0.2 |
| butyl carbamate, PEG-4 | |
| Caprylic/Capric Triglyceride | 0.5-2 |
| Hydroxypropyl Starch phosphate | 0.5-2 |
| Octyldodecanol | 0.1-0.9 |
| DMDM Hydantoin | 0.1-0.9 |
| Ammonium acryloyldimethyltaurate/PV/Copolyme R, | 0.8-3 |
| Trilaureth-4 Phosphate, Polyglyceryl-2-sesquiisostearate | |

When the plant essential oil (e.g. menthol or eucalyptol) was employed to manufacture the antiperspirant composition, the concentration of TRPM8 agonist in said essential oil was measured via GC-MS technique to determine the suitable amount of said essential oil to be used, in order to result in appropriate percentages of TRPM8 agonist in said composition.

### Example 2

### Antiperspirant Effect

The antiperspirant efficacy and safety of investigated compounds were tested pursuant to protocol prescribed by the Food and Drug Administration, 21CFR Part 10.90 (Guidelines for Effectiveness Testing of OTC Antiperspirant Drug Products, 2004). The clinical study further conformed to Normative Resolution 196/96, CN/MS. The clinical study was divided into three tests and conducted with among at least thirty healthy female volunteers per test. They were between the ages of 18 and 50. The study was single-center, randomized, controlled, double blind assessment of the safety, tolerability and antiperspirant efficacy of TRPM8 agonists when applied to the axillae of healthy female volunteers.

Before proceeding with the testing, all the volunteers were submitted to a “rest period” of 17 days, according to the FDA protocol. During this period, a placebo control formulation with no antiperspirancy effect was applied twice a day to both axillae.

Treatments were applied once daily for four consecutive days (numbered D1 to D4). On D1, the baseline perspiration rate was determined before the antiperspirant formulations were applied. All the volunteers remained under hotroom conditions for a 40-minute warm-up period, divided into 2 periods of 20 minutes each. The term “hotroom condition” is the controlled environment (temperatures at 38° C.±1° C., and relative humidity of 35%±5%) established by the FDA standard protocol to thermally induce volunteer's perspiration.

Cotton absorbent pads (Sussex®) were preweighed (P1) and placed in both axillae of each volunteer. At the end of the warm-up period, the perspiration collected on the absorbent pads was again weighed (P2) and the perspiration rates were obtained through gravimetric measurements (according to the formula P2-P1). According to FDA protocol, volunteers must produce at least 100 milligrams of sweat from the placebo control axilla in a 20-minute collection in the controlled environment, and at least one volunteer must produce more than 600 milligrams of sweat.

Commercial menthol (YSC), Mentha essential oil (YSC) and Eucalyptus essential oil (Inter-Link) were presented as components of the antiperspirant formulations as described in the Example 1. Both commercial Mentha and Eucalyptus essential oils, which present menthol and eucalyptol, respectively, as the major components, were submitted to a GC-MS analysis in order to determine the appropriate amount of essential oils to be used to manufacture the antiperspirant compositions.

In each treatment group as listed below, one of the antiperspirant formulations were applied to one axilla of the volunteers. The designation of axillae (namely, right or left) for the application of the antiperspirant formulations was randomized according to the FDA protocol. Treatments were applied once daily for four consecutive days (D1 to D4; D1 after the determination of the baseline perspiration rates).

The study was conducted in three cohorts of volunteers. The groups comprised the following:

Group 1-35 volunteers; one of the antiperspirant formulations was applied in each axilla in a randomized pattern; 40-minute warm-up period one hour after the formulation application.

Group 2-38 volunteers; one of the antiperspirant formulations was applied in each axilla in a randomized pattern; 40-minute warm-up period two hours after the formulation application.

Group 3-30 volunteers, one of the antiperspirant formulations was applied in each axilla in a randomized pattern; 40-minute warm-up period four hours after the formulation application.

The antiperspirant efficacy evaluation was performed one, two and four hours after four days of treatment using standard hotroom protocol. The sweat was collected after each 20-minute warm-up period. The statistical analysis was performed to test antiperspirancy effect, and considered the ratio between the D1 and D4 data. Safety and tolerance were measured by assessment of visual effects and dermal irritancy, and subjective assessment of tolerance. No adverse events occurred during the study.

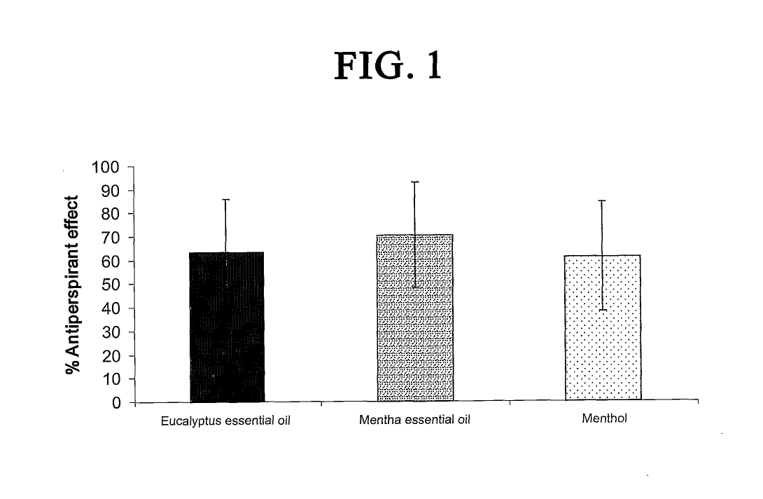

Group 1: The formulations comprising Eucalyptus essential oil (Inter-Link), Mentha essential oil (YSC) and menthol (YSC) showed a statistically significant reduction in sweat production of 63.5%±22.5, 70.7%±22.1 and 61.4%±23.1, respectively (FIG. 1). All three tests met the binomial criteria of 95% confidence with at least 50% of the population showing at least 20% reduction in sweating when compared to the perspiration rate determined at D1. There was no statistical difference between the three tested formulations in terms of antiperspirancy efficacy (p=0.65).

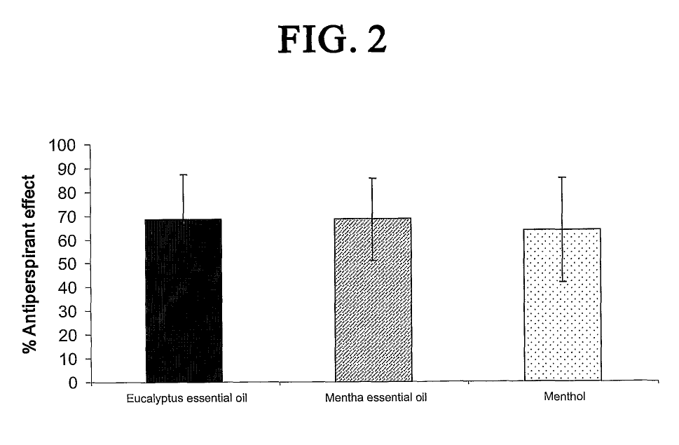

Group 2: The formulations comprising Eucalyptus essential oil (Inter-Link), Mentha essential oil (YSC) and menthol (YSC) showed a statistically significant reduction in sweat production of 68.5%±18.8, 68.4%±17.4 and 63.7%±21.8, respectively (FIG. 2). All three tests met the binomial criteria of 95% confidence with at least 50% of the population showing at least 20% reduction in sweating when compared to the perspiration rate determined at D1. There was no statistical difference between the three tested formulations in terms of antiperspirancy efficacy (p=0.22).

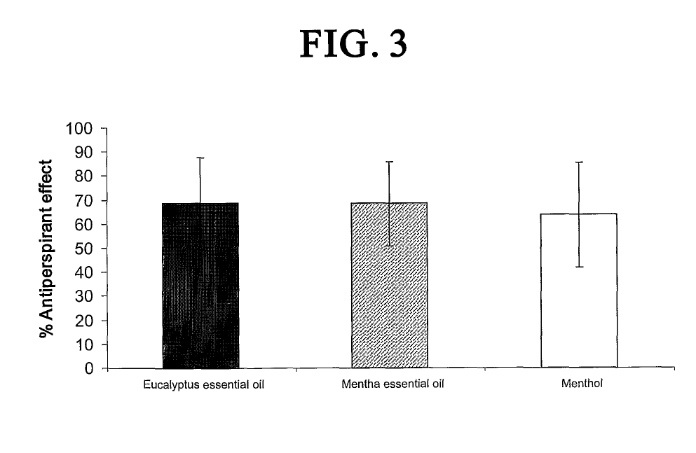

Group 3: The formulations comprising Eucalyptus essential oil (Inter-Link), Mentha essential oil (YSC) and menthol (YSC) showed a statistically significant reduction in sweat production of 72.9%±12.7, 76.5%±13.2 and 75%±12.1, respectively (FIG. 3). All three tests met the binomial criteria of 95% confidence with at least 50% of the population showing at least 20% reduction in sweating when compared to the perspiration rate determined at D1. There was no statistical difference between the three tested formulations in terms of antiperspirancy efficacy (p=0.69).

### Example 3

### Reactivity Evaluation

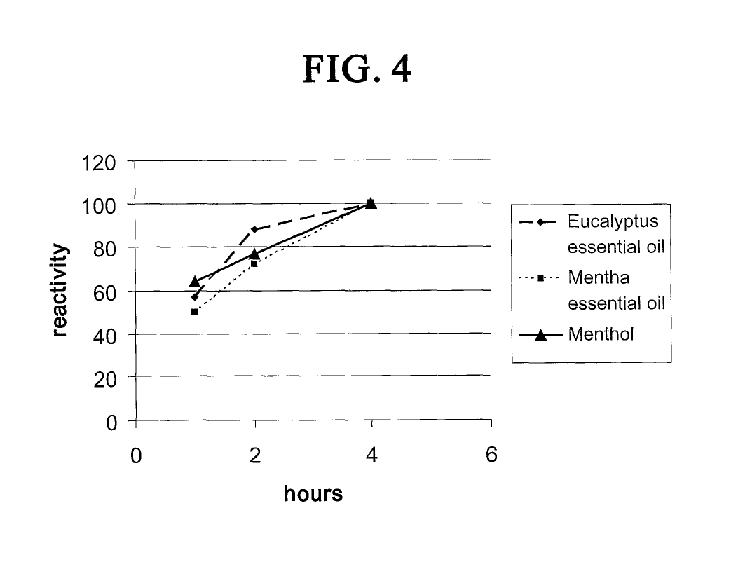

According to FDA Guidelines for Effectiveness Testing of OTC Antiperspirant Drug Products (2004), the standard antiperspirant efficacy is demonstrated when at least 50% of the target population obtains a sweat reduction of at least 20%. On the other hand, extra-effective antiperspirant efficacy is demonstrated when at least 50% of the target population obtains a sweat reduction of at least 30%. The reactivity curve was determined for the agonists tested in our clinical trial, and it was observed that 100% of the target population obtained at least 50% of sweat reduction, 4 h after treatment, as shown in FIG. 4, thus demonstrating that the use of an antiperspirant composition comprising TRPM8 agonist led to an extra-effective antiperspirancy efficacy.

### Example 4

### Evaluation of Deodorant Effect (Sniff Test)

The volunteers were submitted to a “rest period” as described previously in Example 2.

The odor was evaluated for the volunteers at D1 and at D4. At D1 the evaluation was made before the application of the test formulations, after the 20-minute warm-up period, and only the volunteers with starting values not less than 2 were included in the test. The odor evaluation scoring system was based on a range of axilla odor from no malodor (0), to moderate malodor (5), to extremely strong malodor (10). Two trained judges, familiar with the procedures and rating system, subjectively made the odor measurements of both axillae of each volunteer. The data correspond to the individual subject scores by the two trained judges and to the individual average.

At D4, it was observed a statistical significant improvement of 9.7%±25 (p=0.0208), 8.7%±21.6 (p=0.1447) and 6.4%±25.3 (p=0.0018) in axilla odor with the use of the formulations comprising Eucalyptus essential oil (Inter-Link), Mentha essential oil (YSC) and menthol, respectively. There was no statistical difference between the three tested formulations in terms of deodorant efficacy (p=0.08).

The above clinical assays demonstrate the decrease in perspiration achieved by the use of the antiperspirant composition of the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. An antiperspirant composition for topical application to inhibit or reduce perspiration and/or unpleasant body odors, comprising:
a TRPM8 agonist or
a plant essential oil containing a TRPM8 agonist, or
a combination thereof, and
a vehicle,
wherein the TRPM8 agonist is present at a concentration between 0.01% and about 30%, weight per weight, of the composition.

2. The composition according to claim 1, wherein the TRPM8 agonist is selected from the group consisting of menthol, eucalyptol, eugenol, geraniol, linalool, nerolidol, terpineol, carvone, menthone, pulegone, and carvacrol or a combination thereof.

3. The composition according to claim 1, wherein the TRPM8 agonist is menthol or eucalyptol, or a combination thereof.

4. The composition according to claim 1, wherein the plant essential oil containing a TRPM8 agonist is Mentha sp. essential oil or Eucalyptus sp. essential oil.

5. The composition according to claim 1, wherein the TRPM8 agonist is at weight per weight between 0.02% and about 20% of the composition.

6. The composition according to claim 1, wherein the TRPM8 agonist is at weight per weight between 0.05% and about 15%.

7. The composition according to claim 1, wherein the TRPM8 agonist is at weight per weight between 0.1% and about 10%.

8. The composition according to claim 1, wherein the TRPM8 agonist is at weight per weight between 0.2% and about 5% of the composition.

9. The composition according to claim 1, wherein the vehicle comprises a material selected from the group consisting of water, alcohol, an anhydrous solution and a hydrophobic matrix.

10. The composition according to claim 1, wherein the TRPM8 agonist or the plant essential oil containing a TRPM8 agonist is in the microencapsulated form.

11. A method for inhibiting or reducing perspiration and/or body unpleasant odors in an individual comprising a step of topically applying an antiperspirant composition as defined in claim 1.

12. The use of a TRMP8 agonist, or a plant essential oil containing a TRPM8 agonist, or a combination thereof, wherein it is for the manufacture of an antiperspirant product for inhibiting or reducing perspiration and/or unpleasant body odors as defined in claim 1.
